(19) United States
(12) Patent Application Publication
(10) Pub. No.: US 2017/0333181 A1
(43) Pub. Date: Nov. 23, 2017

(54) DUAL ELEMENT ACCOMMODATING INTRAOCULAR LENS DEVICES, SYSTEMS, AND METHODS

(71) Applicant: NOVARTIS AG, BASEL (CH)

(72) Inventors: COSTIN EUGENE CURATU, CROWLEY, TX (US); JOHN ALFRED CAMPIN, SOUTHLAKE, TX (US); JONATHAN D. McCANN, MANSFIELD, TX (US); GEORGE HUNTER PETTIT, FORT WORTH, TX (US)

(21) Appl. No.: 15/159,079

(22) Filed: May 19, 2016

(51) Int. Cl. A61F 2/16

(52) U.S. Cl. CPC A61F 2/1624; A61F 2/16; A61F 2/1605; A61F 2230/0006

(57) ABSTRACT

Disclosed herein is an implantable accommodative IOL device for insertion into an eye of a patient, the device comprising an active element and a passive element. The active element has a first thickness and first refractive index, and the active element comprises an electrically responsive optical lens having variable optical power. The passive element has a second thickness and a second refractive index, and the passive element and the active element are aligned along a central axis extending perpendicularly through a central region of the device. The active element and the passive element comprise individual and separate optical lenses.

### TECHNICAL FIELD

This disclosure relates generally to the field of ophthalmic lenses and, more particularly, to electro-active ophthalmic lenses.

### BACKGROUND

The human eye provides vision by transmitting light through a clear outer portion called the cornea, and focusing the image by way of a crystalline lens onto a retina. The quality of the focused image depends on many factors including the size and shape of the eye, and the transparency of the cornea and the lens. When age or disease causes the lens to become less transparent, vision deteriorates because of the diminished light that can be transmitted to the retina. This deficiency in the lens of the eye is medically known as a cataract. Presently, cataracts are treated by surgical removal of the affected lens and replacement with an artificial intraocular lens (“IOL”). Cataract extractions are among the most commonly performed operations in the world.

In the natural lens, distance and near vision is provided by a mechanism known as accommodation. The natural lens is contained within the capsular bag and is soft early in life. The bag is suspended from the ciliary muscle by the zonules. Relaxation of the ciliary muscle tightens the zonules, and stretches the capsular bag. As a result, the natural lens tends to flatten. Tightening of the ciliary muscle relaxes the tension on the zonules, allowing the capsular bag and the natural lens to assume a more rounded shape. In this way, the natural lens can focus alternatively on near and far objects.

As the lens ages, it becomes harder and is less able to change its shape in reaction to the tightening of the ciliary muscle. Furthermore, the ciliary muscle loses flexibility and range of motion. This makes it harder for the lens to focus on near objects, a medical condition known as presbyopia. Presbyopia affects nearly all adults upon reaching the age of 45 to 50.

One approach to providing presbyopia correction is the use of an ophthalmic lens, such as an IOL. Single focal length or monocular IOLs have a single focal length or single power; thus, single focal length IOLs cannot accommodate, resulting in objects at a certain point from the eye being in focus, while objects nearer or further away remain out of focus. Single focal length IOLs generally do not require power to function properly. An improvement over the single focal length IOL is an accommodating IOL, which can actually change focus by movement (physically deforming and/or translating within the orbit of the eye) as the muscular ciliary body reacts to an accommodative stimulus from the brain, similar to the way the natural crystalline lens focuses. Such accommodating IOLs are generally made from a deformable material that can be compressed or distorted to adjust the optical power of the IOL over a certain range using the natural movements of eye's natural zonules and the ciliary body. In some instances, the accommodative IOL includes an electro-active element that has an adjustable optical power based on electrical signals controlling the element, so that the power of the lens can be adjusted based on the patient's physiologic accommodation demand.

The various components of an electro-active or electrically actuated IOL, however, often create an undesirably large implant that is difficult to implant in the eye through a small incision. A large incision can result in surgical complications such as vision loss secondary to scarring or trauma to ocular tissues. Moreover, an electro-active IOL requires power to function correctly, rendering patients vulnerable to poor visual quality in the case of a non-operational IOL experiencing a power or system failure.

The devices, systems, and methods disclosed herein overcome one or more of the deficiencies of the prior art.

### SUMMARY

In one exemplary aspect, the present disclosure is directed to an implantable accommodative IOL device for insertion into an eye of a patient, the device comprising an active element and a passive element. In one aspect, the active element has a first thickness and first refractive index, and the active element comprising an electrically responsive optical lens having variable optical power. In one aspect, the passive element and the active element are aligned along a central axis extending perpendicularly through a central region of the device. In one aspect, the active element and the passive element comprise individual and separate optical lenses.

In one aspect, a light beam passing through the active element has a phase difference from the light beam passing through the passive element.

In one aspect, the active element is configured to be disposed anterior to the passive element upon insertion into the eye.

In one aspect, the active element is configured to be disposed posterior to the passive element upon insertion into the eye.

In one aspect, the first thickness is different than the second thickness.

In one aspect, the active element is configured to mechanically lock with the passive element.

In one aspect, the active element increases the optical power of the accommodative IOL device when the active element is in a powered state.

In one aspect, the active element and the passive element have the same optical power when accommodative IOL device is in an unpowered state.

In one aspect, the active element and the passive element have matching focal points.

In one aspect, the active element and the passive element are configured for implantation in different regions of the eye.

In one aspect, the accommodative IOL device includes a housing configured to hold electrical components and connections to the active element.

In one aspect, the active element comprises tunable optics technology.

In one aspect, the passive element comprises an optical lens having a static optical power.

In one aspect, a first diameter of the active element is sized to be larger than a second diameter of the passive element.

In one aspect, a light beam passing through the active element has a phase difference from the light beam passing through the passive element. In one aspect, the phase difference provides the implantable IOL device with an extended depth of field.

In another exemplary aspect, the present disclosure is directed to an implantable accommodative IOL device for insertion into an eye of a patient, the device comprising an active region and a passive region. In one aspect, the active region is shaped as a disc having a first thickness and first refractive index, and the active region comprising an electrically tunable lens having variable first optical power. The passive region is shaped as an annular ring disposed circumferentially around the active region, and the passive region comprising an optical lens having a static second optical power. The passive region has a second thickness and a second refractive index. In one aspect, the passive element and the active element are aligned in parallel along a central axis extending perpendicularly through the passive and active elements. In one aspect, a light beam passing through the active element has a phase difference from the light beam passing through the passive element.

In one aspect, the first thickness is different than the second thickness.

In one aspect, the first refractive index is different than the second refractive index.

In one aspect, the active element and the passive element have the same optical power when accommodative IOL device is in an unpowered state.

In one aspect, the active element increases the optical power of the accommodative IOL device when the active element is in a powered state.

In one aspect, the active element and the passive element have the same optical power when accommodative IOL device is in an unpowered state.

In one aspect, the active element and the passive element have matching focal points.

In one aspect, the phase difference provides the implantable IOL device with an extended depth of field.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram of a cross-sectional side view of an eye.

FIG. 2 illustrates a front view of an exemplary accommodative IOL device according to one embodiment consistent with the principles of the present disclosure.

FIG. 3A illustrates a cross-sectional view of an exemplary accommodative IOL device according to another embodiment consistent with the principles of the present disclosure.

FIG. 3B illustrates a cross-sectional view of the exemplary accommodative IOL device shown in FIG. 3A positioned within the eye in a manner consistent with the principles of the present disclosure.

FIG. 3C illustrates a cross-sectional view of the exemplary accommodative IOL device shown in FIG. 3A positioned within the eye in a manner consistent with the principles of the present disclosure.

FIG. 4A illustrates a cross-sectional view of an exemplary accommodative IOL device according to another embodiment consistent with the principles of the present disclosure.

FIG. 4B illustrates a cross-sectional view of the exemplary accommodative IOL device shown in FIG. 4A positioned within the eye in a manner consistent with the principles of the present disclosure.

FIG. 4C illustrates a cross-sectional view of the exemplary accommodative IOL device shown in FIG. 4A positioned within the eye in a manner consistent with the principles of the present disclosure.

FIG. 5 illustrates a schematic view of the exemplary accommodative IOL device shown in FIG. 3A focusing light at a far distance in a manner consistent with the principles of the present disclosure.

FIG. 6 illustrates a schematic view of the exemplary accommodative IOL device shown in FIG. 3A focusing light at a near distance in a manner consistent with the principles of the present disclosure.

FIG. 7 illustrates a perspective view of an exemplary accommodative IOL device according to an embodiment of the present disclosure.

FIG. 8 illustrates a cross-sectional view of the exemplary accommodative IOL device shown in FIG. 7 implanted within the eye according to one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

The present disclosure relates generally to devices, systems, and methods for use in alleviating ophthalmic conditions, including visual impairment secondary to presbyopia, cataracts, and/or macular degeneration. As described above, electrically actuated accommodative intraocular lens (“IOL”) devices have the risk of becoming nonoperational or providing poor visual quality in the case of a power or system failure. Embodiments of the present disclosure comprise accommodating IOL devices configured to correct for far- and/or near-sighted vision and to provide good image quality and extended depth of field (“EDOF”) capabilities even in cases of system failure. In some embodiments, the accommodative IOL devices described herein provide good visual quality by maintaining monofocal vision quality and providing extended depth of field even in an unpowered situation. The accommodative IOL devices described herein are configured to provide clear corrective vision and high image quality to patients having various visual deficits and various pupil sizes.

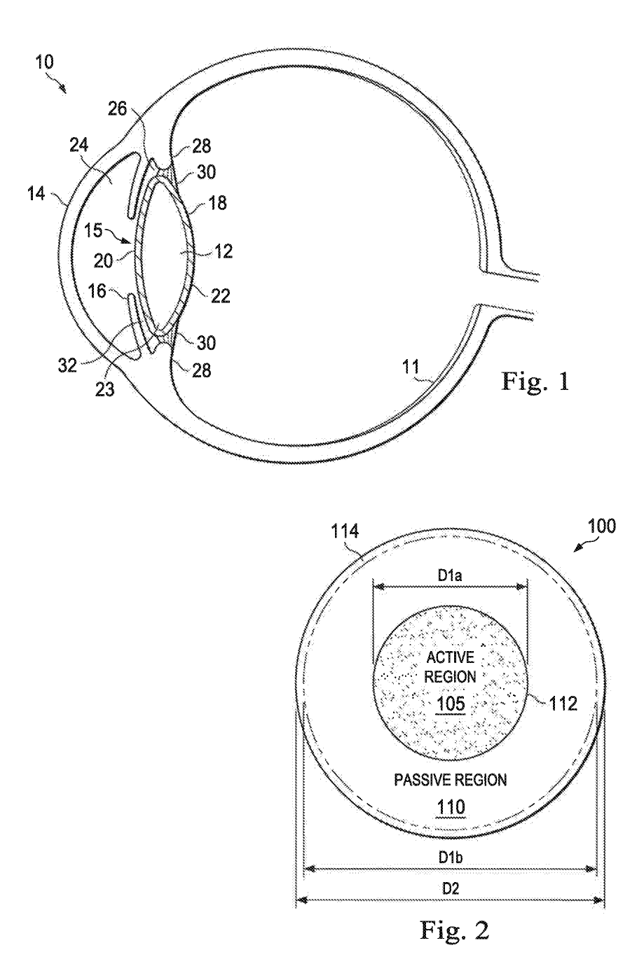

In some embodiments, the accommodating IOL devices described herein include an electro-active optical component and a passive optical component that are separable and distinct parts of the device. Such embodiments may facilitate implantation through a smaller incision than a conventional monolithic electro-active accommodative implant. In some instances, the accommodating IOL devices described herein can be implanted in the eye to replace a diseased lens (e.g., an opacified natural lens of a cataract patient). In other instances, the accommodating IOL devices described herein may be implanted in the eye sulcus 32 (shown in FIG. 1) anterior to the natural lens. In some embodiments, the accommodating IOL devices described herein include multiple optical components that may be configured to complement each other and to cooperate to enhance the patient's vision while being implanted in different regions of the eye. In some embodiments, the embodiments described herein comprise features described in U.S. Provisional application Ser. No. ______ (PAT056413, 45463.460) and Ser. No. ______ (PAT056415, 45463.462), filed ______, which are incorporated by reference herein in their entirety.

FIG. 1 is a diagram of an eye 10 showing some of the anatomical structures related to the surgical removal of cataracts and the implantation of IOLs. The eye 10 comprises an opacified lens 12, an optically clear cornea 14, and an iris 16. A lens capsule or capsular bag 18, located behind the iris 16 of the eye 10, contains the opacified lens 12, which is seated between an anterior capsule segment or anterior capsule 20 and a posterior capsular segment or posterior capsule 22. The anterior capsule 20 and the posterior capsule 22 meet at an equatorial region 23 of the lens capsule 18. The eye 10 also comprises an anterior chamber 24 located in front of the iris 16 and a posterior chamber 26 located between the iris 16 and the lens capsule 18.

A common technique of cataract surgery is extracapsular cataract extraction (“ECCE”), which involves the creation of an incision near the outer edge of the cornea 14 and an opening in the anterior capsule 20 (i.e., an anterior capsulotomy) through which the opacified lens 12 is removed. The lens 12 can be removed by various known methods including phacoemulsification, in which ultrasonic energy is applied to the lens to break it into small pieces that are promptly aspirated from the lens capsule 18. Thus, with the exception of the portion of the anterior capsule 20 that is removed in order to gain access to the lens 12, the lens capsule 18 remains substantially intact throughout an ECCE. The intact posterior capsule 22 provides a support for the IOL and acts as a barrier to the vitreous humor within the vitreous chamber. Following removal of the opacified lens 12, an IOL may be implanted within the lens capsule 18, through the opening in the anterior capsule 20, to restore the transparency and refractive function of a healthy lens. The IOL may be acted on by the zonular forces exerted by a ciliary body 28 and attached zonules 30 surrounding the periphery of the lens capsule 18. The ciliary body 28 and the zonules 30 anchor the lens capsule 18 in place and facilitate accommodation, the process by which the eye 10 changes optical power to maintain a clear focus on an image as its distance varies.

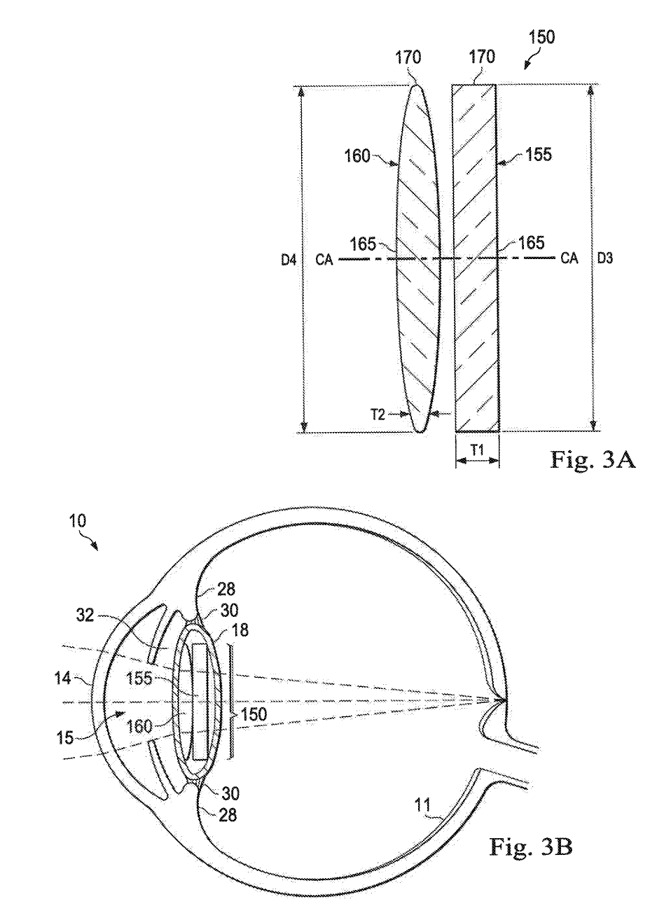

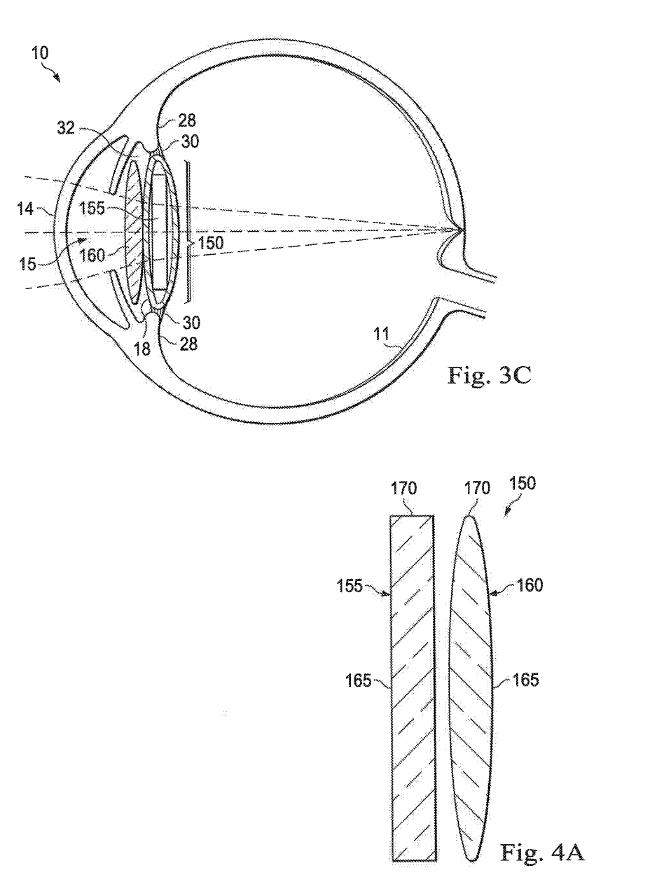

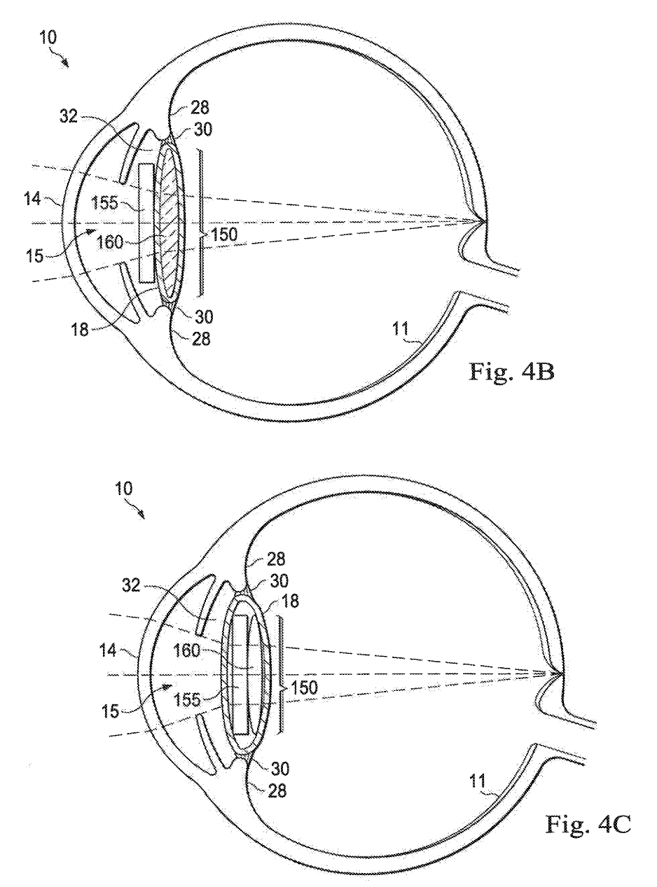

FIG. 2 illustrates a front view of an exemplary accommodative IOL device 100 according to one embodiment consistent with the principles of the present disclosure. The accommodating IOL devices described herein are configured to provide clear vision and accommodation capability using an electro-active or active component in addition to a passive component. In exemplary embodiments disclosed herein, the accommodative IOL device 100 comprises a circular and at least partially flexible disc configured to be implanted in the lens capsule 18 or the eye sulcus 32. As shown in FIGS. 2 and 3, the accommodative IOL device 100 is shaped as a generally circular disc comprising an active region 105 and a passive region 110. In some embodiments, the active region 105 and the passive region 110 comprise a single lens. In other embodiments, for example as shown in FIGS. 3A and 4A, the active region 105 and the passive region 110 form separate optical components that may be shaped and configured to couple together.

In the pictured embodiment, the active region 105 occupies a central region of the IOL device 100, while the passive region 110 extends to a peripheral region of the IOL device 100. The active region 105 is shaped and configured as a generally circular component. In other embodiments, the active region 105 may have any of a variety of shapes, including for example rectangular, ovoid, oblong, and square. In some embodiments, the active region 105 includes a refractive index that is different than the refractive index of the passive region 110. The active region 105 includes a thickness T1 that may range from 0.2 mm to 2 mm. For example, in one exemplary embodiment, the thickness T1 of the active region 105 may be 0.6 mm. In some embodiments, the thickness T1 of the active region 105 varies from the center of the active region 105 to a periphery 112 of the active region 105. For example, in some embodiments, the active region 105 may taper in thickness from its center to its periphery 112.

The electro-active or active region 105 may comprise any of a variety of materials having optical properties that may be altered by electrical control. The active region 105 comprises an electro-active element that can provide variable optical power via any available tunable optics technology including, by way of non-limiting example, moving lenses, liquid crystals, and/or electro-wetting. Although the alterable properties described herein typically include refractive index and optical power, embodiments of the invention may include materials having other alterable properties, such as for example, prismatic power, tinting, and opacity. The properties of the materials may be affected and controlled electrically, physically (e.g., through motion), and/or optically (e.g., through light changes). The active region 105 has an adjustable optical power based on electrical input signals controlling the region, so that the power of the accommodative IOL device 100 can be adjusted based on the patient's sensed or inputted accommodation demand. The accommodative IOL device 100 may include control circuitry, power supplies, and wireless communication capabilities. In some embodiments, this componentry may be packaged in a biocompatible material and/or sealed electronic packaging.

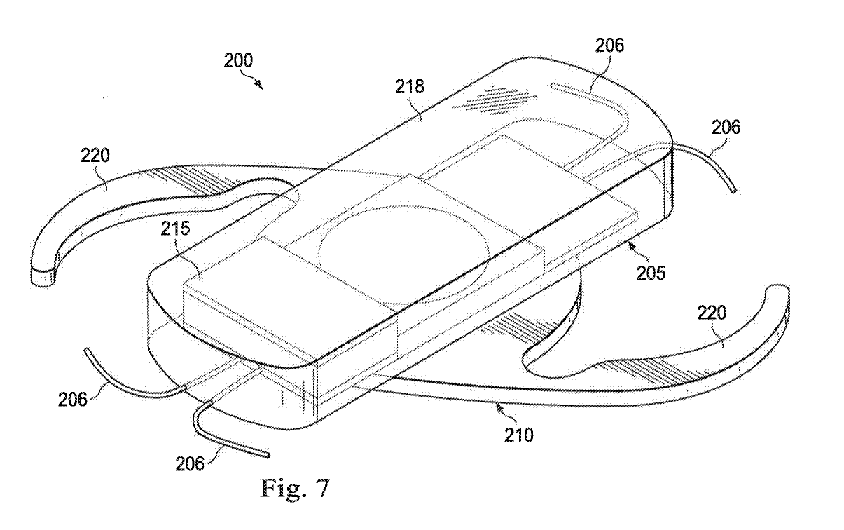

In some embodiments, the passive region 110 is shaped and configured as an annular ring encircling the active region 105. In other embodiments, the passive region 110 is shaped and configured as a separate disc adjacent to the active region 105, as shown in FIG. 3A. In some embodiments, the passive region 110 includes a refractive index that is different than the refractive index of the active region 105. The passive region 110 and the active region 105 are formed from any of a variety of biocompatible materials. In some embodiments, the passive region 110 is formed of relatively more flexible materials than the active region 105. In some embodiments, the active region 105 may be associated with several other components designed to power and control the active region, as shown in FIG. 7. Although the outer diameter D1a of the active region 105 is shown as substantially smaller than an outer diameter D2 of the passive region 110 in the pictured embodiment, the outer diameter D1a of the active region 105 may be sized larger relative to an outer diameter D2 of the passive region 110 in other embodiments. In the pictured embodiment, the active region 105 includes a diameter D1a that is smaller than a diameter D2 of the passive region 110. However, in other embodiments, as indicated by the dotted line, an outer diameter D1b of the active region 105 may be almost as large (or equivalent to) as the outer diameter D2 of the passive region 110. In various embodiments, the outer diameter D1 of the active region 105 may range from 3 mm to 6 mm, and the outer diameter D2 of the passive region 110 may range from 6 mm to 12 mm. For example, in one exemplary embodiment, the outer diameter D1 of the active region 105 may be 3 mm, and the outer diameter D2 of the passive region 110 may be 6 mm.

The accommodative IOL device 100 is designed and optimized to have matching focuses (or matching focal points) for both the active region 105 and the passive region 110 to provide a focused image on the retina 11 for far objects for all pupil sizes. As the object draws closer to the eye 10, the optical power of the active region 105 may be adjusted in response to the input signal (e.g., the electrical input signal) to keep the image focused on the retina 11. This provides accommodation to the patient in a similar manner as a healthy natural crystalline lens.

If the active region 105 cannot be powered due to, by way of non-limiting example, a system failure or an empty battery, the active region 105 is shaped and configured to act like a passive or monofocal lens. In an exemplary embodiment, the unpowered active region 105 has the same optical power as the passive region 110. However, the active region 105 may perform as a passive lens having a different optical power than the passive region 110 (e.g., because of thickness and refractive index differences between the two regions). In particular, the light beams passing through the active region 105 and the light beams passing through the passive region 110 may have a phase difference because of these thickness and refractive index differences. This creates an optical effect similar to the Alcon trapezoidal phase shift lens, which includes optical features described in U.S. Pat. No. 8,241,354, entitled “AN EXTENDED DEPTH OF FOCUS (EDOF) LENS TO INCREASE PSEUDO-ACCOMMODATION BY UTILIZING PUPIL DYNAMICS,” which is incorporated herein by reference. As described in that patent, a linear change in the phase shift imparted to incoming light as a function of radius (referred to herein as a “trapezoidal phase shift”) can adjust the effective depth of focus of the accommodative IOL device 100 for different distances and pupil sizes. This phase difference can be defined as the difference in wavefront in units of waves (Δw):

$$Δ w = \frac{{( {n}_{a} - {n}_{p} )}^{⋆} t}{wavelength}$$

where nₐ is the refractive index of the active region 105, nₚ is the refractive index of the passive region 110, and t is the difference in thickness between the two regions. In this manner, the trapezoidal phase shift provides different apparent depth of focus depending on pupil size, allowing the image to change as a result of changes in light conditions. This in turn provides slightly different images for conditions in which one would be more likely to be relying on near or distance vision, allowing the patient's visual function to better operate under these conditions, a phenomenon known as “pseudo-accommodation.” In particular, the waves having phase differences will interfere, thereby creating extension of the depth of field and a smooth continuity of visual extension.

Thus, a phase difference between the two regions (i.e., the active region 105 and the passive region 110) creates an extended depth of field for the patient that allows the patient to have a range of vision in a situation where the active region 105 cannot receive power or is otherwise malfunctioning. In the case of a system failure or power failure to the active region 105, the accommodative IOL device 100 will continue to have monofocal IOL performance and to provide an extended depth of field to the patient.

FIG. 3A illustrates a cross-sectional view of an exemplary accommodative IOL device 150 according to another embodiment consistent with the principles of the present disclosure. The accommodating IOL device 150 is configured to provide clear vision and accommodation capability using an electro-active or active component in addition to a passive component. The accommodative IOL device 150, like the accommodative IOL device 100 described above, may be used to replace the opacified natural lens 12 of cataract patients and provide the patient with clear vision and enhanced accommodative ability.

As shown in FIGS. 3A and 3B, the accommodative IOL device 150 comprises an electro-active or active element 155 and a passive element 160. Except for the differences described below, the active element 155 may have substantially similar properties to the active region 105 described above with reference to FIG. 2. Except for the differences described below, the passive element 160 may have substantially similar properties to the passive region 110 described above with reference to FIG. 2. Unlike in the accommodative IOL device 100, where the active region 105 and the passive region 110 are part of a single, monolithic optical component, the active element 155 and the passive element 160 of the accommodative IOL device 150 comprise two individual and separable optical components.

As shown in FIGS. 3A-3C, the active element 155 and the passive element 160 form separate optical components or regions that are shaped and configured to function together. In the pictured embodiment, both the active element 155 and the passive element 160 are shaped and configured as generally circular optical components that allow for the passage of light beams through the accommodative IOL device 150 toward the retina 11. In other embodiments, the active element 155 may have any of a variety of shapes, including for example rectangular, ovoid, oblong, and square. In some embodiments, the active element 155 may be associated with several other components designed to power and control the active element, as shown in FIG. 7. Although an outer diameter D3 of the active element 155 is shown as substantially similar to an outer diameter D4 of the passive element 160 in the pictured embodiment, the outer diameter D3 of the active element 155 may be larger or smaller than an outer diameter D4 of the passive element 160 in other embodiments. In particular, the optical performance of embodiments having more flexible active elements 155 may benefit from having active elements 155 that are sized to be larger than the passive elements 160.

The passive element 160 may be shaped and configured to maintain the natural circular contour of the lens capsule 18 and to stabilize the lens capsule 18 in the presence of compromised zonular integrity when the accommodative IOL device 150 is positioned in the eye 10. In some embodiments, the passive element 160 comprises a ring with a substantially circular shape configured to match the substantially circular cross-sectional shape of the lens capsule 18 (shown in FIG. 1) when the lens capsule 18 is divided on a coronal plane through an equatorial region 23.

In some embodiments, the passive element 160 includes a thickness T2 that is different than a thickness T1 of the active element 155. The thickness T1 may range from 0.2 mm to 2 mm. For example, in one exemplary embodiment, the thickness T2 of the active element 155 may be 0.6 mm. The thickness T2 may range from 0.2 mm to 2 mm. For example, in one exemplary embodiment, the thickness T2 of the passive element 160 may be 0.6 mm. In some embodiments, the passive element 160 may taper from a central region 165 towards a peripheral edge 170 of the IOL 150. For example, as shown in FIG. 3A, the thickness T2 of the passive element 160 varies from the center region 165 of the passive element 160 to the peripheral edge 170. In the pictured embodiment, the passive element 160 of the accommodative IOL device 150 comprises atraumatic peripheral edges 170 configured to be positioned within the lens capsule 18 and/or the eye sulcus 32 without inadvertently damaging the lens capsule 18 or other ocular cells.

The peripheral edge 170 comprises the outermost circumferential region of the accommodative IOL device 150. In some embodiments, the accommodative IOL device 150 may taper toward the peripheral edge 170 to facilitate stabilization of the accommodative IOL device 100 inside the lens capsule 18 and/or the eye sulcus 32. This may allow the accommodative IOL device 150 to be self-stabilized and self-retained in the eye 10 (i.e., without the use of sutures, tacks, or a manually held instrument). In some embodiments, the angle of the taper from the passive element 160 towards the peripheral edge 170 is selected to substantially match the angle of the equatorial region 23 in the lens capsule 18, thereby facilitating self-stabilization of the accommodative IOL device 150 within the eye 10.

FIG. 3B illustrates a cross-sectional view of the exemplary accommodative IOL device 150 shown in FIG. 3A positioned within the eye in a manner consistent with the principles of the present disclosure. In the pictured embodiment, the accommodative IOL device 150 comprises an at least partially flexible device configured to be implanted in the lens capsule 18 or the eye sulcus 32 (i.e., the area between the iris 16 and the lens capsule 18). In general, the passive element 160 is relatively more flexible than the active element 155. In one embodiment, the passive element 160 is a large diameter, foldable, relatively soft lens, while the active element 155 is a relatively rigid device having a smaller diameter than the passive element 160.

The two-element accommodative IOL device 150 can reduce the overall incision size during implantation in the eye 10. In particular, the two-element characteristic of the accommodative IOL device 150 allows the surgeon to implant the two lenses (i.e., the active element 155 and the passive element 160) one after another. Each lens or element would have a smaller volume individually than an accommodative IOL device that included both the passive and active elements within a single, monolithic structure. For example, in some instances, the passive element 160 comprises a large diameter, foldable, soft lens and the active element 155 comprises a more rigid, narrower device. Thus, the two-element accommodative IOL device 150 described herein would require a smaller incision than would a monolithic IOL device.

In some embodiments, as shown in FIGS. 3A-3C, the accommodative IOL device 150 may be positioned within the eye such that the active element 155 is positioned posterior to the passive element 160 within the eye 10 (i.e., closer to the anterior chamber 24 of the eye 10). In the pictured embodiment shown in FIGS. 3A-3C, the active element 155 is positioned posterior to the passive element 160. In FIG. 3B, the active element 155 and the passive element 160 are both positioned within the lens capsule 18, but the active element 155 is positioned posterior to the passive element 160. In other embodiments, as shown in FIGS. 4A-4C, the accommodative IOL device 150 may be positioned within the eye 10 such that the active element 155 is positioned anterior to the passive element 160 within the eye 10 (i.e., closer to the anterior chamber 24 of the eye 10). In FIG. 4C, the active element 155 and the passive element 160 are both positioned within the lens capsule 18, but the active element 155 is positioned anterior to the passive element 160.

In other instances, the active element 155 and the passive element 160 are positioned within separate regions of the eye 10. For example, in the embodiment shown in FIG. 3C, the active element 155 is implanted within the lens capsule 18 while the passive element 160 is implanted within the eye sulcus 32. In other instances, as shown in FIG. 4B, the accommodative IOL device 150 is shown implanted within the eye sulcus 32, the area between the iris 26 and the lens capsule 18. In each of these instances, the active element 155 and the passive element 160 are positioned to be aligned along a central axis CA extending perpendicularly through the central region 165 of the device 150.

The active component 155 and the passive component 160 do not necessarily need to be implanted into the eye 10 at the same time. The active component 155 and the passive component 160 may be implanted within the eye 10 sequentially during the same ophthalmic procedure, or may be implanted into the eye 10 in separate procedures, which may occur at different times. In some instances, the active element 155 may be implanted into an eye 10 that already contains a passive lens (e.g., a non-accommodating IOL or a presbyopic natural crystalline lens), thereby offering the possibility of presbyopia correction to a patient that cannot accommodate.

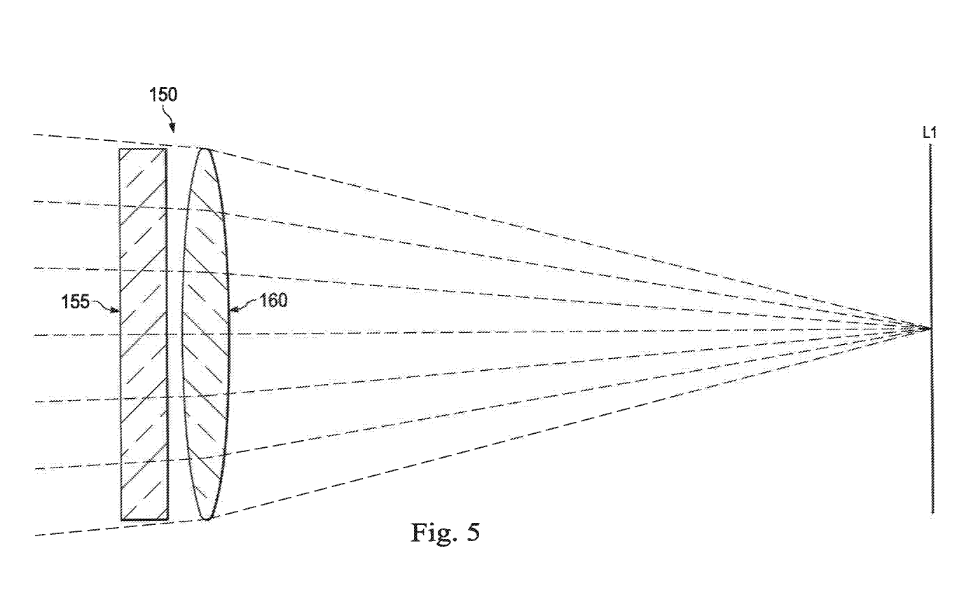

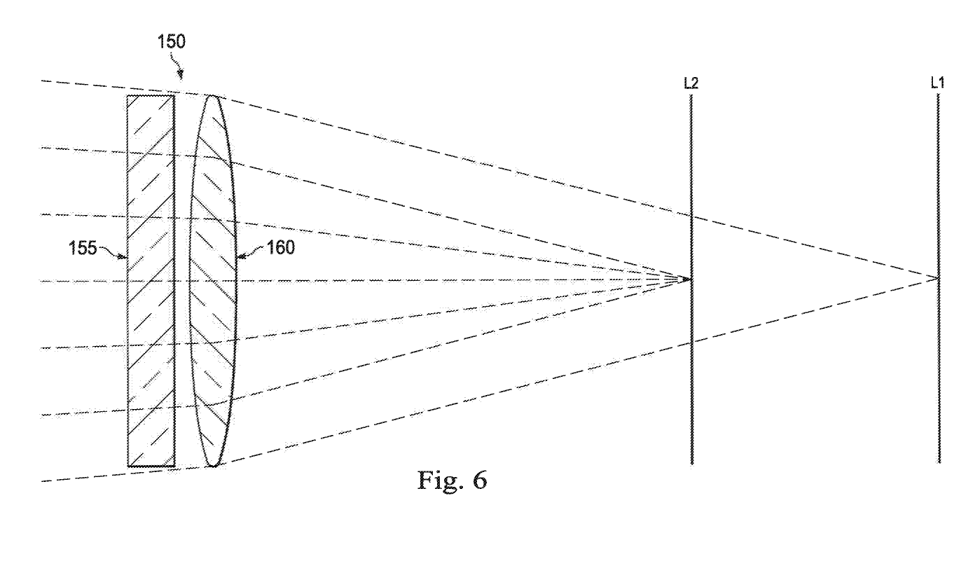

FIG. 5 illustrates a schematic view of the exemplary accommodative IOL device 150 shown in FIG. 3A focusing light at a far distance in a manner consistent with the principles of the present disclosure. FIG. 6 illustrates a schematic view of the exemplary accommodative IOL device 150 shown in FIG. 3A focusing light at a near distance in a manner consistent with the principles of the present disclosure. In some embodiments, the active element 155 provides variable optical power designed mainly to correct for presbyopia, and the passive element 160 provides the static optical power designed mainly to correct refractive error. Thus, as demonstrated in FIGS. 6 and 7, the passive element 160 provides the necessary optical power for the eye to focus at far distances (indicated by the line L1), and the active element 155 provides the additional variable optical power for the eye to be able to focus at all other distances (e.g., a near distance indicated by the line L2). Thus, the active element 155 may remain constant or unchanged for all patients. The individual patient refractive errors as well as other visual aberrations may be corrected with an individually customized passive element 160.

The combination of the two elements—the active element 155 and the passive element 160—is designed to provide the patient with excellent vision at far distances when the IOL is in an unpowered state. When powered, the active element 155 changes the focal length to provide excellent vision for all distances from far to near. For example, if a hypothetical patient needs 25 diopters for excellent far vision, the surgeon may implant an exemplary IOL including a 24 diopter passive element and an active element that has 1 diopter in an unpowered state. When powered, the active element might supply an additional 1 to 3 diopters to provide the patient better near vision. In another instance, the IOL may include a 12.5 diopter passive lens and an active lens having 12.5 diopter optical power when the active lens is unpowered.

By providing unique and separable active and passive optical elements 155 and 160, respectively, the accommodative IOL device 150 allows more options for customizing the combination of accommodative optical power and static optical power and for positioning the elements 155, 160 within the eye 10. In addition, the accommodative IOL device 150 introduces the possibility of implanting only one element of the active and passive elements 155, 160, respectively, into the eye 10. For example, in an instance where the patient has presbyopia without cataracts, it may be preferable to implant only the active element 155 in front of (i.e., anterior to) a non-cataractous, presbyopic crystalline lens.

As mentioned above, the passive element 160 and/or the active element 155 may be shaped and configured to maintain the natural circular contour of the lens capsule 18 and to stabilize the lens capsule 18 in the presence of compromised zonular integrity when the accommodative IOL device 150 is positioned in the eye 10. In some embodiments, the passive element 160 comprises a generally circular disc with a substantially circular shape configured to match the substantially circular cross-sectional shape of the lens capsule 18 when the lens capsule 18 is divided on a coronal plane through an equatorial region 23. In some embodiments, the device 150 (i.e., the active element 155 and/or the passive element 160) may taper from the central region 165 of the device 150 towards the peripheral edge 170. The peripheral edge 170 comprises the outermost circumferential region of the accommodative IOL device 150. In some embodiments, the accommodative IOL device 150 may taper toward its peripheral edge 170 to facilitate stabilization of the accommodative IOL device 100 inside the lens capsule 18 and/or the eye sulcus 32. This may allow the accommodative IOL device 150 to be self-stabilized and self-retained in the eye 10 (i.e., without the use of sutures, tacks, or a manually held instrument). In some embodiments, the accommodative IOL device 150 comprises a substantially circular device having haptic supports 220, as described below in relation to FIG. 7, configured to be self-stabilized within the eye 10 (e.g., within the lens capsule 18 or the sulcus 32). In some embodiments, the angle of the taper from the central region 165 towards the peripheral edge 170 is selected to substantially match the angle of the equatorial region 23 in the lens capsule 18, thereby facilitating self-stabilization of the accommodative IOL device 150 within the eye 10.

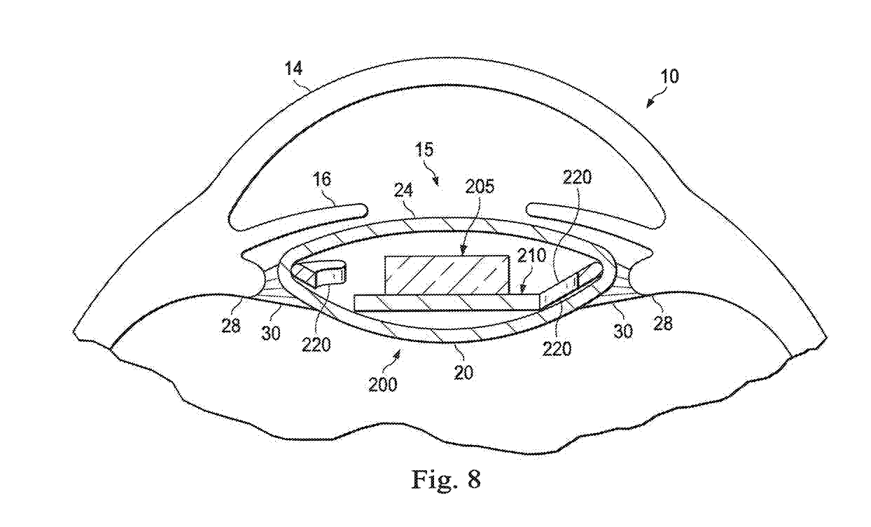

FIG. 7 illustrates a perspective view of an exemplary accommodative IOL device 200 according to one embodiment of the present disclosure. FIG. 8 illustrates a cross-sectional view of the exemplary accommodative IOL device 200 shown in FIG. 7 implanted within the eye 10 according to one embodiment of the present disclosure.

The exemplary accommodative IOL device 200 shown in FIGS. 7 and 8 is substantially the same as the accommodative IOL device 150 shown in FIGS. 3A-6 except for the differences mentioned below. Similar to the accommodative IOL device 150, the accommodative IOL device 200 comprises a two-element IOL including an active component 205 and a passive component 210. The active component 205 is substantially the same as the active element 155 described above. In the pictured embodiment shown in FIG. 7, the accommodative IOL device 200 comprises additional components 215 (e.g., power sources, circuitry, control modules, antennae, etc.) related to the operation of the electro-active element 155. Several of the additional components 215 and the active element 205 are shown gathered together within a housing 218. The passive component 210 is substantially the same as the passive component 160 described above.

In some instances, the two-element accommodative IOL device 200 (and the IOL device 150) can offer enhanced stability of the device and improved protection for the structures of the eye 10 in comparison to conventional IOL devices. For example, in some embodiments, as shown in FIGS. 7 and 8, the passive element 210 may act as an anchoring structure for the active element 205. Moreover, if positioned behind or posterior to the active element 205, the softer passive element 210 can act as a cushion during the implantation procedure of the active element 205 as well as during other procedures such as laser posterior capsulotomies. In some embodiments, the passive and active elements are configured to mechanically lock together (e.g., by snapping into one another or by using a docking mechanism configured to ensure that the two elements are locked together and aligned on a common axis).

In the pictured embodiment, the accommodative IOL device 200 comprises a substantially circular device including haptic supports 220, as shown in FIG. 7, configured to be self-stabilized within the lens capsule 18 of the eye 10 (or the sulcus 32), as shown in FIG. 8. The haptic supports 220 comprise substantially pliable, curved, elongate members extending outwardly from the accommodative IOL device 200. In the pictured embodiment, the haptic supports 220 extend radially from the passive element 210. In other embodiments, the haptic supports 220 may extend from the active element 205. The haptic supports 220 are shaped and configured to expand into the lens capsule 18 and/or the sulcus 32 to stabilize and anchor the IOL device 200 within the eye 10. The haptic supports 220 may be shaped and configured to maintain the natural circular contour of the lens capsule 18 and to stabilize the lens capsule 18 in the presence of compromised zonular integrity when the accommodative IOL device 200 is positioned in the eye 10. In the pictured embodiment, the IOL device 200 includes centralizing members 206 that are shaped and configured to stabilize and centralize the IOL device 200 within the lens capsule 18 of the eye 10 (or the sulcus 32). Other embodiments lack centralizing members 206.

The accommodative IOL devices and systems described herein may be formed from any of a variety of biocompatible materials having the necessary optical properties to perform adequate vision correction as well as requisite properties of resilience, flexibility, expandability, and suitability for use in intraocular procedures. In some embodiments, the individual components of the accommodative IOL devices described herein may be formed of different biocompatible materials of varying degrees of pliancy. For example, in some embodiments, the passive region 110 and the passive elements 160 and 210 may be formed of a more flexible and pliant material than the active region 105 and the active elements 155 and 205 to minimize contact damage or trauma to intraocular structures and to facilitate implantation through a smaller incision. In other embodiments, the reverse relationship may exist. The accommodative IOL devices described herein may be coated with any of a variety of biocompatible materials, including, by way of non-limiting example, polytetrafluoroethylene (PTFE).

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. An implantable accommodative IOL device for insertion into an eye of a patient, the device comprising:
an active element, the active element comprising an electrically responsive optical lens having variable optical power, a first thickness, and a first refractive index; and
a passive element having a second thickness and a second refractive index, the passive element and the active element being aligned along a central axis extending perpendicularly through a central region of the device,
wherein the active element and the passive element comprise individual and separate optical lenses.

2. The accommodative IOL device of claim 1, wherein the active element is configured to be disposed anterior to the passive element upon insertion into the eye.

3. The accommodative IOL device of claim 1, wherein the active element is configured to be disposed posterior to the passive element upon insertion into the eye.

4. The accommodative IOL device of claim 1, wherein the first thickness is different than the second thickness.

5. The accommodative IOL device of claim 1, wherein the active element is configured to mechanically lock with the passive element.

6. The accommodative IOL device of claim 1, wherein the active element and the passive element have the same optical power when accommodative IOL device is in an unpowered state.

7. The accommodative IOL device of claim 1, wherein the active element increases the optical power of the accommodative IOL device when the active element is in a powered state.

8. The accommodative IOL device of claim 1, wherein the active element and the passive element have matching focal points.

9. The accommodative IOL device of claim 1, wherein the active element and the passive element are configured for implantation in different regions of the eye.

10. The accommodative IOL device of claim 1, further comprising a housing configured to hold electrical connections connected to the active element.

11. The accommodative IOL device of claim 1, wherein the active element comprises tunable optics technology.

12. The accommodative IOL device of claim 1, wherein the passive element comprises an optical lens having a static optical power.

13. The accommodative IOL device of claim 1, wherein a first diameter of the active element is sized to be larger than a second diameter of the passive element.

14. The accommodative IOL device of claim 1, wherein a light beam passing through the active element has a phase difference from the light beam passing through the passive element.

15. The accommodative IOL device of claim 14, wherein the phase difference provides the implantable IOL device with an extended depth of field.

16. An implantable accommodative IOL device for insertion into an eye of a patient, the device comprising:
an active region shaped as a disc having a first thickness and first refractive index, the active region comprising an electrically tunable lens having variable first optical power; and
a passive region shaped as an annular ring disposed circumferentially around the active region, the passive region comprising an optical lens having a static second optical power, the passive region having a second thickness and a second refractive index, the passive element and the active element being aligned in parallel along a central axis extending perpendicularly through the passive and active elements,
wherein a light beam passing through the active element has a phase difference from the light beam passing through the passive element.

17. The accommodative IOL device of claim 16, wherein the active element increases the optical power of the accommodative IOL device when the active element is in a powered state.

18. The accommodative IOL device of claim 16, wherein the active element and the passive element have the same optical power when accommodative IOL device is in an unpowered state.

19. The accommodative IOL device of claim 16, wherein the active element and the passive element have matching focal points.

20. The accommodative IOL device of claim 16, wherein the phase difference provides the implantable IOL device with an extended depth of field.
